(19) United States
(12) Patent Application Publication
(10) Pub. No.: US 2026/0297000 A1
(43) Pub. Date: Oct. 1, 2026

(54) SYNGAS PRODUCTION FROM WASTE MATERIALS

(71) Applicant: SemperCycle ApS, Allerød (DK)

(72) Inventor: Jan Kamyno RASMUSSEN, Allerød (DK)

(73) Assignee: SemperCycle ApS, Allerød (DK)

(21) Appl. No.: 18/880,429

(22) PCT Filed: Jun. 30, 2023
(86) PCT No.: PCT/DK2023/000177

§ 371 Date: Dec. 31, 2024

(30) Foreign Application Priority Data
Jul. 1, 2022 (DK) PA 2022 70355

(51) Int. Cl. C07C 29/151; C10J 3/72

(52) U.S. Cl. CPC C07C 29/1518; C10J 3/721; C10J 3/723; C10J 2300/0946; C10J 2300/1665

(57) ABSTRACT

The invention relates to a method for producing syngas from carbonaceous feedstock comprising two or more different compositions of carbonaceous material (e.g., plastics, textiles, biomass, organic matter, natural gas, biogas, carbon dioxide, waste gases), the method comprising: Gasification of the waste feedstock m one or more gasifiers by feeding the feedstock into a gasifier primary reaction zone with a temperature of 500-1000° C., hereby generating: a first output stream; Feeding the first output stream from the first reaction zone into a gasifier secondary reaction zone 80046OQ AC, hereby generating a second output stream comprising the syngas; Feeding the second output stream from the secondary reaction zone into a product synthesis reaction zone, hereby generating a fourth output stream; Separating the fourth output stream from the product reaction into a fifth liquid crude product stream, which is sent for further treatment (e.g., distillation) and at least a sixth and a seventh gas stream; At least part of the sixth gas stream is recycled to the product synthesis reaction zone for further conversion of CO and Hz to the desired product; At least part of the seventh gas: stream Is looped; back to the primary reaction zone; Gasification parameters for the first and the second reaction zones are controlled to take into account the composition and amount of the recycled gas streams.

### AREA OF THE INVENTION

The invention relates to the area of waste handling and more specifically to the area of recycling of waste materials, in particular waste plastics.

### BACKGROUND OF THE INVENTION

End of life materials, often considered waste materials e.g. waste plastics, is a substantial environmental challenge in society. Despite the many good and practical uses of e.g. plastics, the end of life for such uses causes significant pollution and other environmental damage. Even when collected properly the plastics still cause issues due to a plethora of different compositions of the plastics. Often the easiest way of getting rid of the plastics and other waste materials is chosen as the solution, this solution being incineration. The waste does have a certain heating value, however better end of life uses of the material are available. Gasification of waste, e.g. plastics, is a generally well-known process. In such process a gas may be produced that has a number of practical and beneficial uses, that goes beyond the beforementioned incineration and heating.

Waste and in particular plastics is however a more complex composition as a number of different variants are known and used for various purposes.

The gasification of different types of plastics requires different gasification conditions. The complexity in the gasification process appears when gasification of a mixed plastics feedstock is required. Gasification can still be done; however the result of the gasification is often with a less than satisfactory result. Separation of the different plastics types is a cumbersome and time-consuming task and therefore not desirable.

For that reason, there is a need for an improvement in the area of plastics gasification, where a mixed plastics feedstock can be gasified with increased efficiency and hence with a higher yield of the desired gas fraction. By being able to utilize the carbonaceous materials that are normally considered waste materials to produce new and useful materials, the carbonaceous materials now can be considered a valuable resource.

### SUMMARY OF THE INVENTION

According to the invention this is achieved through a method for producing syngas from carbonaceous feedstock comprising two or more different compositions of carbonaceous material (e.g. plastics, textiles, biomass, organic matter, natural gas, biogas, carbon dioxide, waste gases), the method comprising: Gasification of the waste feedstock in one or more gasifiers by feeding the feedstock into a gasifier primary reaction zone with a temperature of 500-1000° C., hereby generating a first output stream; Feeding the first output stream from the first reaction zone into a gasifier secondary reaction zone 800-1600° C., hereby generating a second/third output stream comprising the syngas; Feeding the second/third output stream from the secondary reaction zone into a product synthesis reaction zone, hereby generating a fourth output stream; Separating the fourth output stream from the product reaction into a fifth liquid crude product stream, which is sent for further treatment (e.g. distillation) and at least a sixth and a seventh gas stream; At least part of the sixth gas stream is recycled to the product synthesis reaction zone for further conversion of CO and H₂ to the desired product; At least part of the seventh gas stream is looped back to the primary reaction zone; Gasification parameters for the first and the second reaction zones are controlled to take Into account the composition and amount of the recycled gas streams.

Through such method the carbon is kept longer in the system in order to arrive at the highest yield of desired products when operating with a varying feedstock composition, i.e. reducing the amount of carbon otherwise lost from the process due to inconsistent operating conditions.

The reaction zones may be constituted by individual reactors or may be zones in a reactor designed for multizone operation.

The basis for any adjustment of parameters may be measurements of amount and composition of the recirculated gas streams or may simply be measurements of the changes in the operating conditions incurred by the recirculation and applying a successive correction. Any combination. of those will be possible as well as further control mechanisms that would be adequate.

Advantageously adjusting the gasification parameters for the primary reaction zone (a). includes increasing/decreasing the flow velocity of the second stream in order to provide secondary fuel for the exothermic reaction (to maintain the desired temperature) and/or to maintain the desired total flow velocity in the reaction zone.

Further advantageously adjusting the gasification parameters of the secondary reaction zone (a.) includes increasing/decreasing the flow velocity of the third stream in order to provide secondary fuel for the exothermic reaction (to maintain the desired temperature) and/or to maintain the desired total flow velocity in the reaction zone.

Preferably adjusting the gasification parameters includes maintaining a temperature in the primary reaction zone of 500-1000° C., preferably between 60° and 900° C. and most preferred between 70° and 800° C. by controlling a flow of oxidant and two fuel streams.

Preferably adjusting the gasification parameters includes maintaining a temperature in the secondary reaction zone of 800-1600° C., preferably between 90° and 1500° C. and most preferred between 100° and 1400° C. by controlling a flow of oxidant and two fuel streams.

Advantageously the method includes that the produced syngas is subject to two or more cleaning and conditioning steps, one of them being an addition of externally supplied hydrogen to ensure a CO:H₂ ratio of around 1:2, another one being a compression to e.g. 50-100 bar;

Advantageously the method further includes, that
An eights gas stream can be recycled to the secondary reaction zone for conversion of non-CO or H₂ compounds (such as CHA, C₂+, CO₂ and byproducts from the product reactor) to CO and H₂;
A ninth gas stream can be purged from the system to avoid build-up of inert components (e.g. N₂ and Ar);
A method for producing chemicals (e.g., methanol, Fischer-Tropsch derived compounds, ethylene, propylene) via a syngas produced;
The produced syngas is subject to two or more cleaning and conditioning steps, The conditioned syngas is subject to a conversion in a subsequent reaction zone/reactor to the desired product (e.g., methanol, Fischer-Tropsch derived compounds, ethylene, propylene).

The cleaning and conditioning steps may be incorporated independently of the additional steps mentioned above, preferably between the secondary reaction zone and the product synthesis reaction zone, i.e., second stream providing input to the cleaning and conditioning and the third stream being the output stream providing the input to the product reaction synthesis zone.

Preferably the method includes that one or more of the following parameters are monitored forming a basis for the adjustment of the gasification parameters: T1, Temperature of reaction zone 1, T2, Temperature of reaction zone 2, y1, CO content at outlet of reaction zone 1 (molar/volume flow), y2, CO₂ content at outlet of reaction zone 1 (molar/volume flow), y3, CO content at outlet of reaction zone 2 (molar/volume flow), y4, CO₂ content at outlet of reaction zone 2 (molar/volume flow), y5, Hz content at outlet of reaction zone 2 (molar/volume flow), y6, N₂+Ar content in recycle gas (molar/volume flow).

Preferably the method further includes that one or more of the following input streams are monitored and controlled in order to optimize the gasification parameters: u1, Plastic waste feedstock flow, u2, Oxygen flow to reaction zone 1, u3, Steam flow to reaction zone 1, u4, Recycle gas flow to reaction zone 1, u5, Oxygen flow to reaction zone 2, u6, Recycle gas flow to reaction zone 2, u7, Hydrogen gas flow to product reaction zone/reactor, u8, Recycle gas flow to flare/exhaust.

Preferably the fluidization flow in reaction zone 1, u2+u3+u4, is kept within a predefined Interval (minimum fluidization velocity to minimum blow-out velocity), and where, If the fluidization flow drops below the minimum threshold: Increase 4, or if the fluidization flow goes above the maximum threshold: decrease u4.

Preferably the reaction temperature in reaction zone 1, T1, is kept within a range of 500 to 1000° C., preferably 600 to 900° C., most preferred between 700 and 800° C. and where If T1 drops below a lower predetermined value: decrease u1, increase u2, decrease u3, increase u4, or if T1 goes above an upper predetermined value: increase u1, decrease u2, increase u3, decrease u4.

Preferably the CO/CO₂ ratio (y1/y2) is maintained in a predetermined interval and If y1/y2 drops below the threshold (too much oxidant): increase u1, decrease u2, decrease u3, increase u4, or if y1/y2 goes above the threshold (too less oxidant): decrease u1, increase u2, increase u3, decrease u4.

Preferably the reaction temperature of reaction zone 2, T2, is maintained in a predetermined range of 800 to 1600° C., preferably in the range 900 to 1500° C. and most preferred in the range 1000 to 1400° C. and where if T2 drops below a predetermined lower value: increase u5, increase u6, or if T2 goes above a predetermined upper value: decrease u5, decrease u6.

Preferably the CO/CO₂ ratio (y3/y4) from reaction zone 2 indicates whether the level of oxidant should be adjusted, it should be within a certain predetermined interval and if y3/y4 drops below the threshold (too much oxidant): decrease u5, increase u6, or if y3/y4 drops below the threshold (too less oxidant): increase u5, decrease u6.

Preferably the (H2−CO₂)/(CO+CO₂) ratio (module) should be kept around 2.1, e.g. 2.0-2.2, in the methanol synthesis process, and where the measurements made at the outlet of reactor 2 is used to calculate the required addition of H₂ and where the added hydrogen, u7, is calculated from: (H2−CO₂)/(CO+CO₂)=2.1 ⇔(y5+u7−y4)/(y3+y4)=2.1.

Preferably the level of inert gases, y6 (e.g., nitrogen and argon), is maintained at a low level by controlling the purge gas to flare/exhaust, u8, and if y6 drops below the threshold: decrease u8, or if y6 goes above the threshold: increase u8.

The invention will be described in detail in the following with reference to the figures showing details of a preferred embodiment of the invention.

### LIST OF FIGURES

FIG. 1 shows a system for production of methanol according to the invention;

FIG. 2 shows in more detail a part of a system for production of methanol according to the invention;

FIG. 3 shows in more detail a part of a system for production of methanol according to the invention;

FIG. 4 shows in more detail a part of a system for production of methanol according to the invention;

FIG. 5 shows in more detail a part of a system for production of methanol according to the invention;

FIG. 6 shows a system for production of methanol according to the invention;

### DETAILED DESCRIPTION OF PREFERRED EMBODIMENTS

Feedstock may as mentioned be of a variety of different types. Despite this fact the following description focus on plastics as the main feedstock.

Further, although the desired end product may be methanol as described in the following, the syngas produced may be used for a number of purposes. The following description therefore is only to be regarded an example of implementation of the invention.

### Feedstock Quality

The feedstock composition in terms of the contained components is based on data from a major waste supply company in Denmark. The analysis of each component has been considered to develop an overall analysis of the mixed feed as shown in Table 0-1. This analysis may be used to develop the design case mass and energy balance. Alternative compositions, in particular of significantly larger amounts of PVC, PET, Organics, Inorganics and Moisture may be a reality that will need to be accommodated in a specific design.

TABLE 0-1
Feedstock Analysis

| | Content | | | | | | | | |
|---|---|---|---|---|---|---|---|---|---|
| Component | % | C | N | H | O | Cl | S | Ash | Sum |
| PP | 30 | 84.09 | 0.07 | 14.78 | 1.06 | | | | 100 |
| LDPE | 15 | 85.45 | 0.50 | 13.95 | 0.10 | | | | 100 |
| HDPE | 15 | 85.60 | 0.50 | 13.00 | 0.90 | | | | 100 |
| PVC | 2 | 38.10 | | 4.76 | | 57.14 | | | 100 |
| PET | 15 | 62.50 | 0.57 | 4.17 | 32.76 | | | | 100 |
| PS | 8 | 91.45 | 0.86 | 7.17 | 0.52 | | | | 100 |
| Organics | 5 | 60.70 | 1.20 | 8.20 | 28.70 | 0.95 | 0.25 | | 100 |
| Inorganics | 5 | | | | | | | 100.00 | 100 |
| Moisture | 5 | | | 11.11 | 88.89 | | | | 100 |
| Sum | 100 | 71.37 | 0.39 | 10.74 | 11.30 | 1.19 | 0.01 | 5.00 | 100 |

The heating value has been calculated from published values as shown in Table 0-2.

TABLE 0-2
Heating value of Feedstock

| | Content | LHV |
|---|---|---|
| Component | % | MJ/kg |
| PP | 30 | 42.427 |
| LDPE | 15 | 38.945 |
| HDPE | 15 | 34.105 |
| PVC | 2 | 18.000 |
| PET | 15 | 21.285 |
| PS | 8 | 39.137 |
| Organics | 5 | 16.000 |
| Inorganics | 5 | 0.000 |
| Moisture | 5 | 0.000 |
| Sum | 100 | 31.169 |

Ash quality data are provided in Table 0-3. This is an indicative analysis of likely ash. It is not used in the simulation. Its importance is only in the expectation of an ash fusion temperature of the order of magnitude of 1100-1200° C.

TABLE 0-3
Ash Quality

| | | CaO + | | Na2O+ | | |
|---|---|---|---|---|---|---|
| SiO2 | A12O3 | MgO | FeO | K2O | Other | Sum |
| 50 | 15 | 25 | 5 | 2 | 3 | 100 |

For the feeding system, it is important to consider the effect of temperature on the plastics, Typical data for the glass temperature (T_{g}), the Heat Deflection Temperature (HDT) and the melting point (Tₘ) are given in Table 0-4. The deflection temperature is a measure of a polymer's ability to bear a given load at elevated temperatures. While this temperature is in principle arbitrarily defined, it gives an indication of the potential for a plastic to soften and under the force of a feeding system risk blockages at higher temperatures.

TABLE 0-4
Temperature Properties of Plastic

| | T_{g} | HDT | Tₘ |
|---|---|---|---|
| | ° C. | ° C. | ° C. |
| PP | 0 | 100 | 160 |
| LDPE | −125 | 40 | 110 |
| HDPE | −120 | 85 | 130 |
| PVC | 60 | 92 | 100-260 |
| PET | 67-81 | 70 | 260 |
| PS | 100 | 95 | 270 |

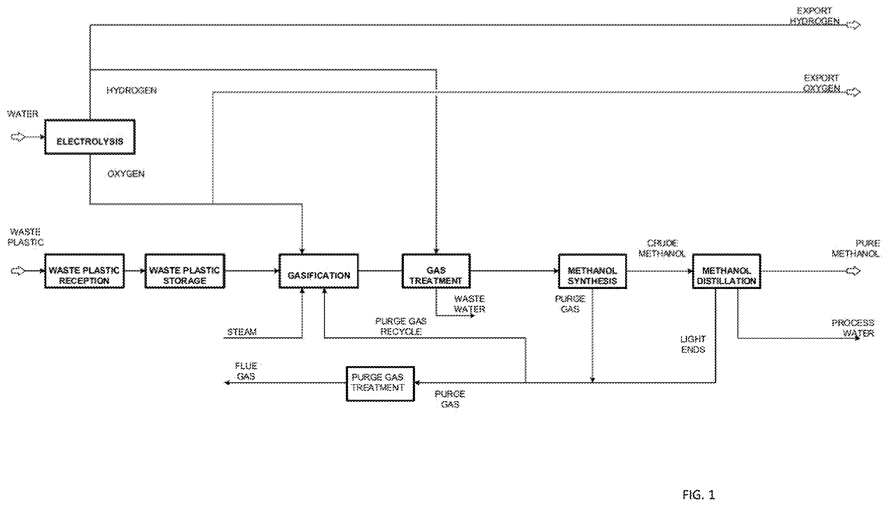

The following description of the FIGS. 1-6 includes details related to a particular embodiment with a particular size and capacity. Whereas this constitutes a preferred embodiment at the time of the application the individual component as well as the scaling of the individual components and the total system may vary from this description and should as such not be considered a limitation for future developments, where the principles of the invention may still be applicable.

From FIG. 1 a schematic diagram shows the main elements of a system for performing the method according to the invention. The system comprises a waste plastics reception system and a waste plastics storage, a gasification system, a gas treatment system, a methanol synthesis system, a methanol distillation system and a electrolysis system.

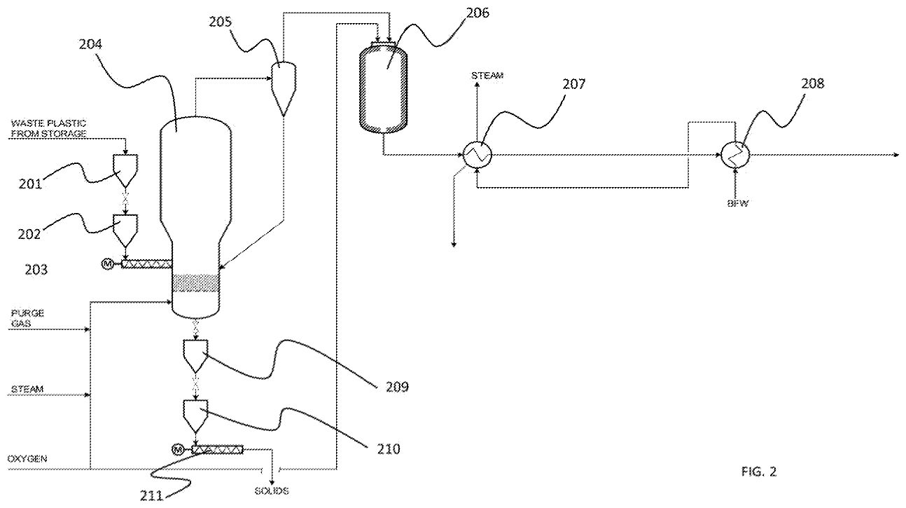

From FIG. 2 a partial view of the system depicted in FIG. 1 is shown in more detail. FIG. 2 shows the gasification system having:
201: Feedstock hopper
202: Lock hopper
203: Screw feeder
204: Primary reactor/primary reaction zone (fluidized bed reactor)
205: Cyclone
206: Secondary reactor/secondary reaction zone (partial oxidation reactor)
207: Waste heat boiler
208: Boiler Feedwater Preheater
209: Ash hopper
210: Ash lock hopper
211: Solids discharge screw

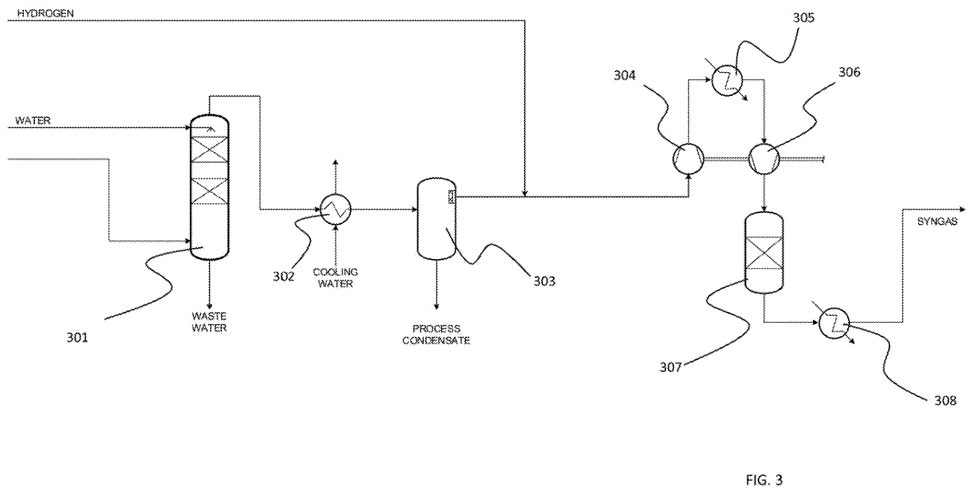

From FIG. 3 a partial view of the system depicted in FIG. 1 is shown in more detail. FIG. 3 shows the gas treatment system with:
301: Raw gas scrubber
302: Raw gas cooler
303: Raw gas separator
304: Syngas compressor, first stage
305: Syngas cooler
306: Syngas compressor, second stage
307: Guard bed or absorption unit
308: Syngas cooler

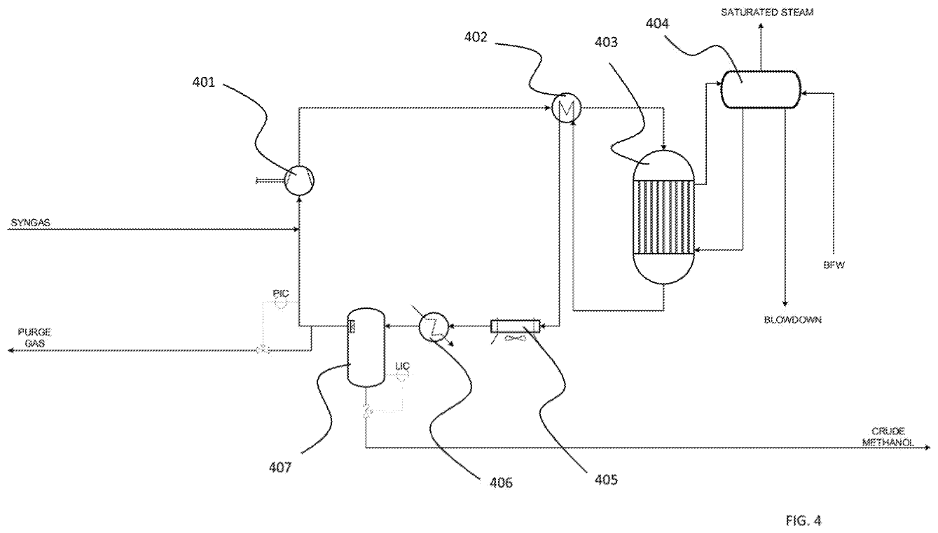

From FIG. 4 a partial view of the system depicted in FIG. 1 is shown in more detail. FIG. 4 shows the methanol synthesis system comprising:
401: Syngas compressor, recirculation stage
402: Heat exchanger
403: Methanol reactor vessel
404: Steam drum
405: Air cooler
406: Water cooler
407: Separator

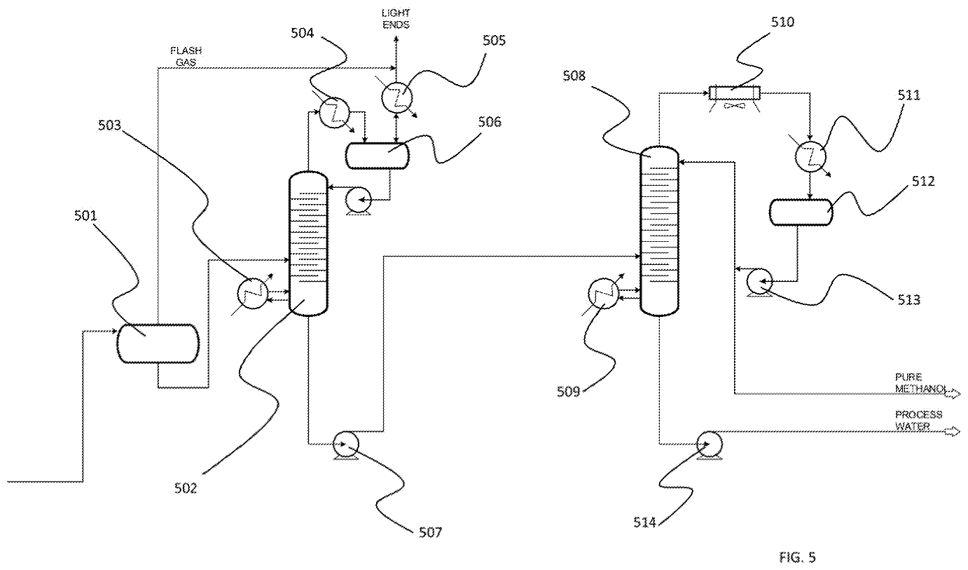

From FIG. 5 a partial view of the system depicted in FIG. 1 is shown in more detail. FIG. 5 shows the methanol distillation system comprising:
501: Flash vessel
502: Light ends distillation column
503: Reboiler
504: Reflux water cooler
505: Light ends cooler
506: Reflux drum
507: Methanol pump
508: Distillation column
509: Reboiler
510: Reflux air cooler
511: Reflux water cooler
512: Reflux drum
513: Pure methanol reflux pump
514: Process water pump

### Overall Process Description

The overall process is performed in a plant as schematically shown in FIG. 1 and as described above. The following description describes the function of the process blocks and their relationship to one another. This is then followed by a description of the individual units where appropriate.

It is assumed that the waste plastic is delivered by road to a reception hall and mechanical pre-treatment. In the reception hall any material deemed unacceptable after a visual inspection is removed. Typically, this could include oversize material, car batteries or the like, In the mechanical pre-treatment (MPT) the feedstock is shredded and sorted to make it suitable for the gasifier and its feeding system. Sorting could for example include ferrous and non-ferrous metal rejection systems. The pre-treated feedstock is then delivered to a buffer store ready for feeding to the gasifier. The buffer store is designed to allow separate storage of different qualities of feedstock, so that a stable mixture can be fed to the gasifier.

### Oxygen and Hydrogen Supply

Oxygen and hydrogen are supplied from an electrolysis plant, preferably supplied with electricity from renewable sources. The oxygen and hydrogen requirements of the plant are nearly in balance. On the basis of the material balance, there will be surplus of the total oxygen supplied. Hydrogen need not be produced on site and could be provided through a pipeline from a remote electrolysis facility or transported in batches and stored locally.

### Gasification

Reference is made to FIG. 2. The gasifier system is an oxygen-fired stationary fluid bed first stage gasifier followed by an entrained flow secondary gasifier to remove tars. An essentially nitrogen-free syngas is required, so oxygen-firing has been selected. In principle a dual fluid bed system would achieve this also but has the disadvantage of emitting CO₂ from the combustor bed. The high temperature secondary gasifier has the advantage of reducing the methane content of the syngas and is able to make use of the oxygen infrastructure of the oxygen-fired primary gasifier.

### Process Description

The whole syngas train upstream the compressor must be under positive pressure to prevent accidental air ingress. The feeding system must allow for this and lock hoppers with a screw feeder are proposed for this duty. Note that the screw feeder may well need cooling, particularly at the front end (closer to the gasifier) to avoid softening of the plastic and potential blocking of the feeder.

The primary gasifier is a stationary fluid bed operating at a temperature of 750° C. The plastics are gasified in the bed, but it must be assumed that some material will vaporize without full conversion to Hz and CO leaving some tars in the gas exiting the gasifier. Solids are drawn off at the bottom of the reactor together with some of the bed material. Bed material and solids can be at least partially separated, and the recovered bed material recycled to the reactor. Fine solids, which may include a small amount of unconverted carbon, will be carried out of the reactor at the top of the freeboard. This will be captured in a cyclone and recycled to the bed.

The secondary gasifier is an entrained flow reactor operating in a similar manner to a partial oxidation (POX) reactor at a temperature of around 1200° C., which is assumed high enough to ensure conversion of all the higher hydrocarbons still contained in the primary gasifier effluent. The gas is then cooled in a waste heat boiler generating saturated steam.

Additional cooling of the gas is performed in the Boiler Feedwater Preheater.

### Gas Treatment

Reference is made to FIG. 3. The gas leaving the Boiler Feedwater Preheater still contains any chlorine originating from PVC in the feedstock. In the formal mass balance this is calculated to present as 0.21 mol % HCL. Below the water dewpoint of about 70° C. HCl corrosion can be expected. But apart from this, there is also the potential for ammonium chloride formation from the ammonia formed during gasification. This must be removed at a higher temperature, typically in the range 170-190° C. by washing with water, which also removes the hydrochloric acid from the gas. The gas is therefore scrubbed with cold water to remove chlorine compounds and it leaves the scrubber at about 70° C. This ensures that the gas is either dry (before entering the scrubber) or the chlorine compounds are sufficiently diluted in the scrubber water that corrosion risks are avoided. The gas leaving the scrubber is water saturated but chlorine-free.

The scrubber also removes any ammonia and HCN formed in the gasifier.

Final cooling to 40° C. is achieved with cooling water and process condensate is removed in a separator.

The gas at this point has a hydrogen: carbon monoxide ratio of slightly over 1 and a CO₂ content of about 12%. For the methanol synthesis hydrogen must be added to achieve an optimum value of just over 2 for the stoichiometric ratio (H2−CO₂)/(CO+CO₂). Hydrogen from the electrolysis unit co-produced with the oxygen is taken to accomplish this.

Fresh syngas is compressed and fed to a desulphurization vessel and/or guard bed or other absorption unit to absorb the H₂S, COS and other non-desired components in the gas.

### Methanol Synthesis

Correct adjustment of the hydrogen injection will allow the production of a synthesis gas with an optimum value of just over 2 for the stoichiometric ratio (H2−CO₂)/(CO+CO₂). The selection of the exact pressure to run the methanol synthesis loop will depend on an OPEX/CAPEX optimization. For larger plants a pressure of 100 bar or so is common, for smaller plants 50 bar is proposed. The basic principles are shown in FIG. 4.

The cleaned and conditioned gas is fed to the suction side of the loop gas circulator, which recycles unconverted syngas from the methanol reactor on the same shaft as the syngas compressor. The gas is preheated in the Feed-Effluent-Exchanger and fed to a tubular reactor in which the carbon oxides and hydrogen are converted to methanol. The tubes are filled with catalyst, which is cooled by the boiling water on the outside of the tubes.

The steam pressure of about 40 bar maintains the desired gas outlet temperature of 250° C. The unconverted gas together with the methanol leaves the reactor and is cooled successively in the Feed-Effluent-Exchanger, an air-cooler and a final water cooler, thus condensing the methanol. The crude methanol is separated out in a separator and fed to the distillation section. Inert gases (mainly methane and nitrogen) are purged from the loop before the remaining gas is fed back to the circulator. The purge gas also contains some H₂ and CO.

### Methanol Distillation

The Distillation Unit Is shown In FIG. 5. The crude methanol contains a small amount of low-boiling co-formed products such as DME as well as some physically dissolved gases. The dissolved gases are flashed off in a Flash Vessel and low boiling impurities (light ends) removed in a Light Ends Column. The stabilized methanol is then distilled in the Atmospheric Column to obtain a specification product. The process water produced as the distillation bottoms stream contains various co-produced impurities such as ethanol.

As an alternative, the distillation can take place in a three-column system to reduce the reboiler steam demand. The quality of the methanol is not influenced by such change.

### Fuel Gas System

Fuel gas is produced in the form of the methanol synthesis purge and the light ends removed in the distillation unit. Approximately 80 wt % of this is recycled to the gasifier, which allows for the contained carbon still to be converted to methanol. The remaining 20 wt % is combusted in a furnace used to superheat the saturated steam generated in the process. This also allows for the removal of inert gases such as nitrogen from the system.

This 80:20 split between recycle and combustion is to some extent arbitrary. This ratio causes a 4-times increase In the nitrogen flowing around the system, but the small amount entering the system means that this is still acceptable. The amount of gas combusted provides suitable superheat (~320° C.) to ensure that the turbine exhaust is still dry.

### Tank Farm

Suitable provision will need to be made for storage of product methanol. An intermediate raw methanol tank will also be required to maintain a stable flow to the distillation unit in the event of fluctuations in the upstream plant including the energy supply to the electrolysis unit.

### Environmental Issues

### Solid Discharges

Ash will be discharged from the gasifier. This is expected to contain up to about 5% carbon. The allowable carbon-in ash for disposal or onward sale needs to be checked on a project basis.

### Liquid Discharges

There are three major liquid discharge streams
scrubber wastewater containing up to 1000 ppm chlorides, as well as traces of ammonia, HCN. H2S and soot.
process condensate containing traces of dissolved gases, mainly CO₂.
process water from the distillation. The main contaminant is ethanol, but other hydrocarbons (e. g. ketones) are also present.

### Process Controls

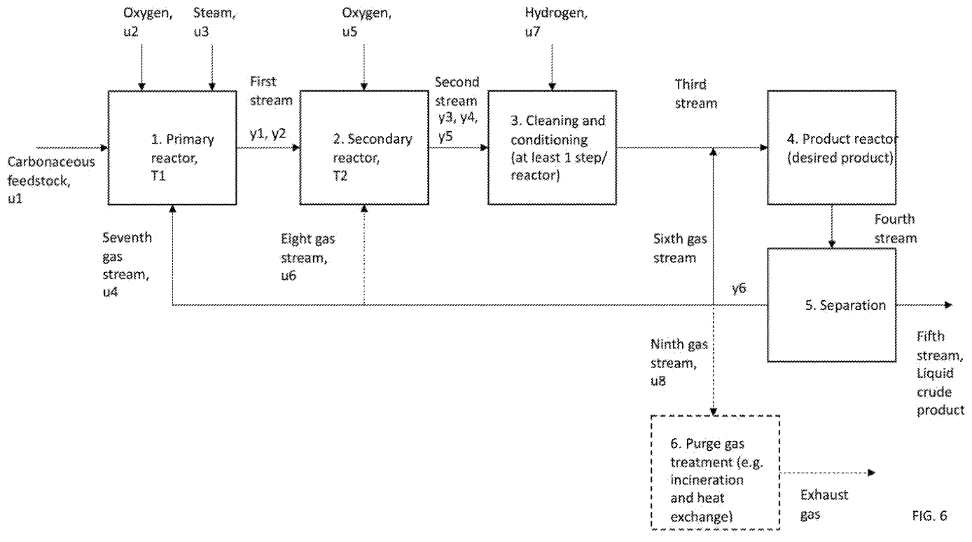

Controlling the process under stationary conditions and with essentially one feedstock type will correspond to the well-known state of the art technology. However, in order to accommodate a higher degree of feedstock flexibility and at the same time a high yield and efficiency of the process, independent of the desired end product, and according to the invention, a recycling of certain gas streams and corresponding adaptation of the process conditions is applied. Reference is made to FIG. 6 indication the measurements and input streams forming part of the most important control loops as explained in the following.

### Overall System

The aim is to design a system which can provide a very high conversion of carbon-containing feedstock of fluctuating and unknown composition to desired product (e.g. methanol).

To achieve this ideally all carbon in the feedstock must be converted to syngas and only exit the system as desired product (e.g., methanol). It will be very difficult to predict the composition of the gas from the reactor and therefore the system has to be robust towards these fluctuations. An essential element is the utilization of controlled recycle loops which allow for conversion of unconverted carbon containing gases to syngas.

The system is preferably run on renewable (fluctuating) electricity providing energy for water electrolysis. Optionally a connection to a hydrogen-pipeline could be considered, this allowing for a remote location of an electrolysis facility. Even though the produced hydrogen and oxygen will be stored as a buffer to avoid too much ramping up and down (complete shut-downs due to lack of green electricity should be avoided) it might be beneficial to add a certain degree of modulation in the design.

A typical industrial methanol plant would have a stable flow of well-defined feedstock and the need for recycle loops would not be needed.

In this context syngas is defined as a gas consisting primarily of CO, H₂, CO, In various amounts.

### Reactor 1:

This is a fluidized bed type reactor operating at around 750° C. and a slight over pressure (around 0-1 bar). As the reactor is of the fluidized bed type the gas flow entering the reactor should be in a certain interval to “lift” the bed material and ensure fluidization (minimum fluidization velocity) and avoid “blow-out” of the bed material. The fluidized bed has good mass and energy transfer properties and can be compared to a well-mixed liquid. The bed material is typically quartz sand or olivine and might have limited catalytic effects on syngas formation.

The plastic waste feedstock enters the side of the reactor slightly above the top of the fluidized region. As the density of the feedstock is higher than of the gas in the reactor it will drift down and enter the fluidized bed. Light particles with a large surface will have a very short residence time in the fluidized bed before they are converted to gas and the opposite goes for heavy particles with small surface.

All gas enters the bottom of the reactor, flows through the bed and leaves the reactor in top. A cyclone receives this gas and separates solid particles out which are directed back to the fluidized bed via a separate tube. The gas, which is now almost free of solid particles, exits the cyclone and enters reactor 2.

The gas entering the reactor is composed of 3 separate gas flows:
1. Oxygen
2. Steam
3. Recycle gas from the methanol reactor

The recycle gas will contain unconverted CO, CO₂ and H₂ from the methanol reactor as well as species entering the methanol reactor which it cannot convert, e.g. CH₄ and byproducts from the methanol reaction (e.g., ethanol and DME). The recycle gas can have varying compositions but will have a high energy content and can act as a secondary feedstock for the primary reactor.

No air is supposed to enter the system as nitrogen and argon will act as inert gases which have to be purged from the system. A small amount of air will enter via the feeding system.

The intention of reactor 1 is to perform partial oxidation, steam reforming and cracking reactions with the purpose of bringing all feedstock to a gas phase which will leave the reactor and be converted further in reactor 2. The aim is not to perform a full conversion to syngas in the first reactor. The gas at the exit of reactor 1 is expected to contain primarily CO, H₂, CO₂ and some amounts of CH₄, C₂_{⁺} (incl tar), HCl, H₂S and NH₃ as well as other species.

The key reactions performed in reactor 1 are as follows:

$$Depolymerization$$

$$[ {CH}_{2} ] n = n / 2 {C}_{2} {H}_{4} {ΔH}_{r}^{0} = + 60 kJ / mol$$

$$Partial oxidation$$

$$1 / 2 {C}_{2} {H}_{4} + 1 / 2 {O}_{2} = CO + {H}_{2} {ΔH}_{r}^{0} = - 137 kJ / mol$$

$$Steam reforming$$

$$1 / 2 {C}_{2} {H}_{4} + {H}_{2} O ( g ) = CO + 2 {H}_{2} {ΔH}_{r}^{0} = + 105 kJ / mol$$

$$Steam methane reforming$$

$${CH}_{4} + {H}_{2} O ( g ) = CO + 3 {H}_{2} {ΔH}_{r}^{0} = + 206 kJ / mol$$

$$Reverse water gas shift$$

$${CO}_{2} + {H}_{2} = CO + {H}_{2} O ( g ) {ΔH}_{r}^{0} = + 41 kJ / mol$$

$$Reverse Bouduard reaction$$

$${CO}_{2} + C = 2 CO {ΔH}_{r}^{0} = + 172 kJ / mol$$

$$Hydrogen combustion$$

$${H}_{2} + 1 / 2 {O}_{2} = {H}_{2} O ( g ) {ΔH}_{r}^{0} = - 242 kJ / mol$$

A fraction of the bed material is continuously taken out in the bottom of the reactor and filtered or replaced by new bed material. Inorganic matter (such as metal parts, fillers, sand and gravel) will either be taken out in the gas phase or taken out as slag mixed with bed material in the bottom of the reactor.

The reactor can be considered autothermic as it balances exothermic reactions (e.g. partial oxidation) with endothermic reactions (e.g. steam reforming) to keep a desired temperature (e.g. 750° C.) in the fluidized bed. The reactor is highly insulated and mainly exchanges energy with the material flows (gas in, gas out, feed in).

The temperature of 750° C. has been chosen as a compromise between conversion rate and material specifications. The reactor is not lined which means that the metal must be suited to withstand the temperature. A higher temperature will give a better conversion to syngas but will restrain the selection of possible metal alloys. A lower temperature will give lower conversion to syngas, higher formation of tars (e.g. PAH's) and carbon formation (coking) due to the Bouduard reaction which shifts CO to C and CO₂ at lower temperatures.

The reactor will be controlled by monitoring temperatures and gas composition, the controlled input will be waste plastic feed flow, oxygen flow, steam flow and recycle gas flow.

The temperatures will be measured in the bed and above the bed. The gas analyzers will measure CO, CO₂ and H₂ in the outlet.

### Reactor 2:

This is a partial oxidation reactor operating at e.g., 1200° C. It will most likely be a refractory lined tubular reactor with gas feed in one end and gas exit at the other end. It will be fed with the gas from the cyclone after reactor 1 as well as oxygen and optionally recycle gas. Exothermic partial oxidation reactions will convert CH₄, C₂_{⁺}, tars and other unconverted carbon species to CO and H₂. A certain residence time (probably less than a second) at this temperature is required to ensure that all tars are converted. Tar conversion is very important as it can lead to clogging in the cleaning step and fouling of the methanol catalyst.

In this design a non-catalytic reactor has been chosen but in principle a catalytic reactor (catalytic tar reformer) could be used. The catalytic reactor could be fully or partially heated by electromagnetic induction. This would have the advantage of a lower temperature, e.g. 900-1000° C., but the disadvantage would be higher CAPEX and the risk of catalyst deactivation and additional maintenance.

In principle steam could be added as and oxidizing agent in combination with the oxygen, but as the reaction is endothermic this would require even more oxygen or electrical energy to keep the desired temperature. Steam would have the advantage of producing more Hz than oxygen and thus increasing the H2:CO ratio.

The addition of oxygen should be sufficient to raise the temperature to the desired level (e.g. 1200° C.) and provide enough oxygen for conversion of carbon containing species to CO. A lack of oxygen could result in uncomplete tar conversion. On the other hand, too much oxygen would convert some of the CO to CO₂, which is a “waste” of energy and CO for the methanol synthesis. In reality some CO has to be “sacrificed” in the process.

The reactor will be controlled by monitoring temperatures and gas composition, the controlled input will be the oxygen flow and optionally recycle flow.

The temperatures will be measured in the inlet, outlet and possibly somewhere in-between. The gas analyzers will measure CO, CO, and H, In the inlet, outlet and possibly somewhere in-between.

### Cleaning and Conditioning:

One or more unit operations are used to clean and condition the syngas. Most likely a scrubber (possibly alkaline) will be used to remove HCl, NH₃, other N-containing compounds, and most other impurities. A subsequent de-sulphurization unit and possibly guard-bed might be added to remove sulphur and other potential catalyst poisons. It is very important that catalyst poisons like Cl, Br, F and S are brought down to very low levels, e.g. ppb according to the catalyst manufacturers specifications. Otherwise the methanol catalyst will quickly deactivate.

Before the cleaned syngas enters the methanol loop, the syngas is compressed to 50-100 bar and H₂ is added to achieve a (H2−CO₂)/(CO+CO₂) ratio of around 2.1 which is considered optimal for methanol production.

This is a simple control system monitoring the CO, CO₂ and H₂ composition, the controlled input being the flow of added H₂

### Methanol (Product) Reactor:

The compressed syngas enters the methanol reactor, which contains methanol catalyst and is controlled to a temperature of around 225° C. by cooling water. As the conversion to methanol of each pass of the gas is relatively low (around 10% of the syngas) the unconverted syngas is looped back the reactor for another pass. This happens a number of times and the recycle flow in the methanol loop is much larger than syngas inflow. After each pass a methanol separator separates gas and liquid (crude methanol).

Instead of methanol, a Fischer-Tropsch reaction could be used or in the future a direct syngas-to-olefins reaction might become commercialized. The loop is essentially the same.

### Separation:

The methanol separator mentioned above separates the unconverted syngas, incl. species which have entered the reactor which cannot be converted (e.g. CH₄ and C₂+) and byproducts from the methanol reaction (e.g. ethanol and DME).

The methanol separator is in principle just a drum where equilibrium between the gas and liquid phase is established. The crude methanol contains a significant amount of water (e.g. 30%) and is distilled in subsequent distillation process.

The majority of the gas from the methanol separator is looped back to the methanol reactor, but a significant fraction is recycled to the primary and/or secondary reactor and small fraction is purged.

The recycle to reactor 1 has the purpose of:
1. Converting species which cannot be converted in the methanol reactor, such as CH₄, C₂_{⁺}, ethanol and DME, to syngas;
2. Provide additional fuel to the reactor to increase the temperature (e.g. in case of low-calorific feedstock);
3. Provide additional gas flow to the reactor to ensure sufficient fluidization (e.g. in the case of low system utilization/feedstock flow due to lack of green electricity).

The recycle to reactor 2 has the purpose of:
1. Converting species which cannot be converted in the methanol reactor, such as CH₄, C₂_{⁺}, ethanol and DME, to syngas;
2. Provide additional fuel to the reactor to increase the temperature (e.g., if the tar content is not sufficient to provide the required temperature—this way less CO would have to be “sacrificed”).

### Purge Gas Treatment:

Only a small part of the gas is expected to be purged. The purpose of this is to reduce the amount of inert gases (like N₂ and Ar) in the system.

This will take place in a furnace or flare where the gas will be incinerated at a high temperature to reduce emissions of e.g. dioxins and furans.

Ideally the energy should be recovered and used for steam generation or steam superheating.

### Most Important Control Loops

### Controlled Inputs:

u1, Plastic waste feedstock flow
u2, Oxygen flow to reactor 1
u3, Steam flow to reactor 1
u4, Recycle gas flow to reactor 1
u5, Oxygen flow to reactor 2
u6, Recycle gas flow to reactor 2
u7, Hydrogen gas flow to methanol reactor
u8, Recycle gas flow to flare/exhaust
The total recycle gas flow from the methanol separation unit will be equal to: u4+u6+u8+recycle flow to the methanol reactor

The control system will be relatively complicated and might be constructed as a number of feedback/feedforward control loops (e.g. PID controllers) or ideally a model predictive control (MPC) system based on a mathematical model of the system.

Monitored outputs:
T1, Temperature of reactor 1
T2, Temperature of reactor 2
y1, CO content at outlet of reactor 1 (molar/volume flow)
y2, CO₂ content at outlet of reactor 1 (molar/volume flow)
y3, CO content at outlet of reactor 2 (molar/volume flow)
y4, CO₂ content at outlet of reactor 2 (molar/volume flow)
y5, H₂ content at outlet of reactor 2 (molar/volume flow)
y6, N₂+År content in recycle gas (molar/volume flow)

In the control loop examples in the following, specific conditions have been chosen, which should only be considered examples. The invention may be implemented with conditions varying from the exemplified conditions and as specified in the claims.

### Control Loop 1: Maintain Desired Fluidization Flow of Reactor 1

### Constraint:

The fluidization flow, u2+u3+u4, must be within a defined interval (minimum fluidization velocity to minimum blow-out velocity).

### Control Logic:

If the fluidization flow drops below the minimum threshold: increase u4
If the fluidization flow goes above the maximum threshold: decrease u4

### Control Loop 2: Maintain Desired Temperature of Reactor 1

### Constraint:

The reactor temperature, T1, must be in an interval around 750° C. (e.g. 740-760° C.).

### Control Logic:

If T1 drops below 740° C.: decrease u1, increase u2, decrease u3, increase u4
If T1 goes above 760° C.: increase u1, decrease u2, increase u3, decrease u4

This controls the amounts of exothermic and endothermic reactions and ensures process stability despite varying feedstock compositions.

Control Loop 3: Maintain Desired Gas Composition from Reactor 1

### Constraint:

The CO/CO₂ ratio (y1/y2) indicates whether the level of oxidant should be adjusted, it should be in a certain interval.

### Control Logic:

If y1/y2 drops below the threshold (too much oxidant): increase u1, decrease u2, decrease u3, increase u4
If y1/y2 goes above the threshold (too less oxidant): decrease u1, increase u2, increase u3, decrease u4

### Control Loop 4: Maintain Desired Temperature of Reactor 2

### Constraint:

The reactor temperature, T2, must be in an interval around 1200° C. (e.g. 1150-1250).

### Control Logic:

If T2 drops below 1150° C.: increase u5, increase u6
If T2 goes above 1250° C.: decrease u5, decrease u6
   Control Loop 5: Maintain Desired Gas Composition from Reactor 2

### Constraint:

The CO/CO₂ ratio (y3/y4) indicates whether the level of oxidant should be adjusted, it should be in a certain interval.

### Control Logic:

If y3/y4 drops below the threshold (too much oxidant): decrease u5, increase u6
If y3/y4 goes above the threshold (too less oxidant): increase u5, decrease u6

### Control Loop 6: Maintain Desired Gas Composition for Methanol Reactor

### Constraint:

The (H2−CO₂)/(CO+CO₂) ratio (module) should be kept around 2.1, e.g. 2.0-2.2. The measurements made at the outlet of reactor 2 can be used to calculate the required addition of H₂ as the gas composition should not change during the cleaning process.

### Control Logic:

The added hydrogen, u7, can be calculated from:

$$( H 2 - {CO}_{2} ) / ( CO + {CO}_{2} ) = 2.1 \text{?} ( y 5 + u 7 - y 4 ) / ( y 3 + y 4 ) = 2.1$$

### Control Loop 7: Maintain Level of Inert Gases

### Constraint:

The level of inert gases, y6 (e.g. nitrogen and argon), should be maintained at a low level by controlling the purge gas to flare/exhaust, u8

### Control Logic:

If y6 drops below the threshold: decrease u8
If y6 goes above the threshold: increase u8

1. A method for producing syngas from carbonaceous feedstock comprising two or more different compositions of carbonaceous material, the method comprising:
a. Gasification of the waste feedstock in one or more gasifiers by feeding the feedstock into a gasifier primary reaction zone with a temperature of 500-1000° C., hereby generating a first output stream;
b. Feeding the first output stream from the first reaction zone into a gasifier secondary reaction zone 800-1600° C., hereby generating a second output stream comprising the syngas;
c. Feeding the second output stream from the secondary reaction zone into a product synthesis reaction zone, hereby generating a fourth output stream;
d. Separating the fourth output stream from the product reaction into a fifth liquid crude product stream, which is sent for further treatment and at least a sixth and a seventh gas stream;
e. At least part of the sixth gas stream is recycled to the product synthesis reaction zone for further conversion of CO and H₂ to the desired product;
f. At least part of the seventh gas stream is looped back to the primary reaction zone;
g. Gasification parameters for the first and the second reaction zones are controlled to take into account the composition and amount of the recycled gas streams.

2. A method according to claim 1, where adjusting the gasification parameters for the primary reaction zone (a). includes increasing/decreasing the flow velocity of the fourth stream in order to provide secondary fuel for the exothermic reaction (to maintain the desired temperature) and/or to maintain the desired total flow velocity in the reaction zone.

3. A method according to claim 1, where adjusting the gasification parameters of the secondary reaction zone includes increasing/decreasing the flow velocity of the eights gas stream in order to provide secondary fuel for the exothermic reaction (to maintain the desired temperature) and/or to maintain the desired total flow velocity in the reaction zone.

4. A method according to claim 1, where adjusting the gasification parameters includes maintaining a temperature in the primary reaction zone of 500-1000° C., preferably between 60° and 900° C. and most preferred between 70° and 800° C. by controlling a flow of oxidant and two fuel streams

5. A method according to claim 1, where adjusting the gasification parameters includes maintaining a temperature in the secondary reaction zone of 800-1600° C., preferably between 90° and 1500° C. and most preferred between 100° and 1400° C. by controlling a flow of oxidant and two fuel streams.

6. A method according to claim 1, where the produced syngas is subject to two or more cleaning and conditioning steps, one of them being an addition of externally supplied hydrogen to ensure a CO:H₂ ratio of around 1:2, another one being a compression to 50-100 bar;

7. A method according to claim 1, where adjusting the gasification parameters includes
h. A eight gas stream can be recycled to the secondary reaction zone for conversion of non-CO or H₂ compounds to CO and H₂;
i. A ninth gas stream can be purged from the system to avoid build-up of inert components;
j. A method for producing chemicals via a produced syngas;
k. The produced syngas is subject to two or more cleaning and conditioning steps,
l. The conditioned syngas is subject to a conversion in a subsequent product reaction zone/reactor to the desired product.

8. A method according to claim 1, where one or more of the following parameters are monitored forming a basis for the adjustment of the gasification parameters: T1, Temperature of reaction zone 1, T2, Temperature of reaction zone 2, y1, CO content at outlet of reactor 1 (molar/volume flow), y2, CO₂ content at outlet of reaction zone 1 (molar/volume flow), y3, CO content at outlet of reaction zone 2 (molar/volume flow), y4, CO₂ content at outlet of reaction zone 2 (molar/volume flow), y5, H₂ content at outlet of reaction zone 2 (molar/volume flow), y6, N₂+Ar content in recycle gas (molar/volume flow).

9. A method according to claim 1, where one or more of the following input streams are monitored and controlled in order to optimize the gasification parameters: u1, Plastic waste feedstock flow, u2, Oxygen flow to reaction zone 1, u3, Steam flow to reaction zone 1, u4, Recycle gas flow to reaction zone 1, u5, Oxygen flow to reaction zone 2, u6, Recycle gas flow to reaction zone 2, u7, Hydrogen gas flow to methanol reactor, u8, Recycle gas flow to flare/exhaust.

10. A method according to claim 9, where the fluidization flow in reaction zone 1, u2+u3+u4, is kept within a predefined interval (minimum fluidization velocity to minimum blow-out velocity), and where, if the fluidization flow drops below the minimum threshold: increase u4, or if the fluidization flow goes above the maximum threshold: decrease u4.

11. A method according to claim 9, where the reaction temperature in reaction zone 1, T1, is kept within a range of 500 to 1000° C. and where If T1 drops below a lower predetermined value: decrease u1, increase u2, decrease u3, increase u4, or if T1 goes above an upper predetermined value: increase u1, decrease u2, increase u3, decrease u4.

12. A method according to claim 9, where CO/CO₂ ratio (y1/y2) is maintained in a predetermined interval and If y1/y2 drops below the threshold (too much oxidant): increase u1, decrease u2, decrease u3, increase u4, or if y1/y2 goes above the threshold (too little oxidant): decrease u1, increase u2, increase u3, decrease u4.

13. A method according to claim 9, where the reaction temperature of reaction zone 2, T2, is maintained in a predetermined range of 800 to 1600° C. and where if T2 drops below a predetermined lower value: increase u5, increase u6, or if T2 goes above a predetermined upper value: decrease u5, decrease u6.

14. A method according to claim 9, where the CO/CO₂ ratio (y3/y4) from reaction zone 2 indicates whether the level of oxidant should be adjusted, it should be within a certain predetermined interval and if y3/y4 drops below a lower threshold (too much oxidant): decrease u5, increase u6, or if y3/y4 goes above an upper threshold (too little oxidant): increase u5, decrease u6.

15. A method according to claim 9, where the (H2−CO₂)/(CO+CO₂) ratio (module) should be kept around 2.1, e.g. 2.0-2.2, in the methanol synthesis process, and where the measurements made at the outlet of reactor 2 is used to calculate the required addition of H₂ and where the added hydrogen, u7, is calculated from: (H2−CO₂)/(CO+CO₂)=2.1⇔(y5+u7−y4)/(y3+y4)=2.1.

16. According to claim 9, where the level of inert gases, y6, is maintained at a low level by controlling the purge gas to flare/exhaust, u8, and if y6 drops below a lower threshold: decrease u8, or if y6 goes above an upper threshold: increase u8.

17. A method according to claim 2, where adjusting the gasification parameters includes maintaining a temperature in the primary reaction zone of 500-1000° C. by controlling a flow of oxidant and two fuel streams

18. A method according to claim 3, where adjusting the gasification parameters includes maintaining a temperature in the primary reaction zone of 500-1000° C. by controlling a flow of oxidant and two fuel streams

19. A method according to claim 2, where adjusting the gasification parameters includes maintaining a temperature in the secondary reaction zone of 800-1600° C. by controlling a flow of oxidant and two fuel streams.

20. A method according to claim 3, where adjusting the gasification parameters includes maintaining a temperature in the secondary reaction zone of 800-1600° C. by controlling a flow of oxidant and two fuel streams.
